(19) United States
(12) Patent Application Publication
(10) Pub. No.: US 2016/0184495 A1
(43) Pub. Date: Jun. 30, 2016

(54) SURFACE PUMP AND DRESSING COMPRISING A SURFACE PUMP

(71) Applicant: Commissariat a l'energie atomique et aux energies alternatives, Paris (FR)

(72) Inventors: Yves FOUILLET, Voreppe (FR); Olivier CONSTANTIN, Grenoble (FR)

(73) Assignee: Commissariat a l'energie atomique et aux energies alternatives, Paris (FR)

(21) Appl. No.: 14/982,508

(22) Filed: Dec. 29, 2015

(30) Foreign Application Priority Data
Dec. 30, 2014 (FR) 14 63432

(51) Int. Cl. A61M 1/00; F04B 53/10; F04B 43/12

(52) U.S. Cl. CPC A61M 1/0023; F04B 43/12; F04B 53/10

(57) ABSTRACT

The invention relates to a surface pump comprising a network (3) of microfluidic channels (5) and pumping means (7). Said network of microfluidic channels includes a set of microfluidic accesses (9) distributed in a predetermined domain (11) suited to covering a surface source of a fluid of interest, and said pumping means comprise at least one microfluidic actuating circuit (11) intersecting said network of microfluidic channels on a plurality of deformable membrane intersecting zones (17) forming a set of valves (19) suited to causing a peristaltic effect pumping of said fluid of interest circulating in said network of microfluidic channels from said set of microfluidic accesses to at least one sink (21) connected to said network of microfluidic channels.

### TECHNICAL FIELD

The present invention relates to the general field of microfluidic drainage of a liquid from a surface source and, more particularly, the drainage of exudates secreted by a wound. The invention pertains to a surface pump as well as a dressing for exudative wounds comprising a surface pump.

### STATE OF THE PRIOR ART

Generally speaking, channels, suction pads or porous materials connected to pumps are used to suck up a liquid on a surface.

More particularly, to drain or suck up liquids produced by wounds, negative pressure drainage systems are used. In fact, acute or chronic wounds secrete liquids called exudates, the control of which is an important problem which care teams are faced with. Thus, the treatment of the wound consists in part in removing the exudates.

The document WO 03/086232 describes an example of a system for treating wounds comprising a negative pressure dressing. The dressing comprises a layer in contact with the wound, including a central zone in communication with a vacuum pump and a plurality of channels diverging radially from the central zone and including holes to extract the exudate from the wound under the effect of a negative pressure.

In order to create a negative pressure, the dressing necessarily has to be leak tight which complicates its application on the wound and requires continuous surveillance and upkeep. Furthermore, the vacuum pump is bulky and noisy.

Moreover, the suction takes place in an overall manner over the whole surface of the dressing. This poses a problem of drainage in the case where certain zones are dry and not others. In fact, the suction effect will be accentuated on the dried out zone which does not need any drainage while preventing the correct drainage of exudates secreted by the other zones.

Moreover, the exudate is evacuated to a bag external to the dressing via fluidic connections which further increase the bulk of the system. The connections are obviously soiled by the exudate and can potentially pollute the pump.

The purpose of the present invention is, consequently, to overcome the aforementioned drawbacks by proposing an efficient surface pumping system with a simple connection architecture and more particularly a dressing with surface pumping for exudative wounds, simple to apply, hygienic and having reduced bulk.

### DESCRIPTION OF THE INVENTION

The aim of the invention is to propose a compact surface pump having a high efficiency of drainage of a liquid on a surface whatever the heterogeneity of the humidity level or the spreading of the liquid. Another aim of the invention is to offer a dressing with efficient and hygienic surface pumping for exudative wounds enabling simplified application, and having a simple connection architecture and low bulk.

The subject matter of the invention is a surface pump comprising a network of microfluidic channels and pumping means. Said network of microfluidic channels includes a set of microfluidic accesses distributed in a predetermined domain suited to covering a surface source of a fluid of interest, and said pumping means comprise at least one microfluidic actuating circuit intersecting said network of microfluidic channels on a plurality of deformable membrane intersecting zones forming a set of valves suited to causing a peristaltic effect pumping of said fluid of interest circulating in said network of microfluidic channels from said set of microfluidic accesses to at least one sink connected to said network of microfluidic channels.

Thus, the surface pump is compact, easy to implement even though the number of valves may be very high (for example of the order of several hundred), and operates in an optimal manner to pump the liquid on several paths which can cover a large surface, and to do so whatever the heterogeneity of the spreading of the liquid on the surface.

According to a preferred embodiment of the present invention, said network of microfluidic channels comprises a set of separate microfluidic drainage channels provided with said set of microfluidic accesses, each microfluidic drainage channel being associated with a determined group of valves, and the surface pump comprises a single microfluidic actuating circuit suited to being connected to a single pressure generator, said actuating circuit being constituted of resistive and capacitive fluidic elements defining a response time delay between two successive valves belonging to a group of valves, said response time delay being configured to cause, under an action of said pressure generator, a sequential actuation of the valves.

Thus, the surface pump only requires a single connection path connected to a single actuating source, further optimising its compactness and its simplicity.

Advantageously, said set of microfluidic drainage channels comprises a set of main microfluidic channels and a plurality of secondary microfluidic channels, each microfluidic drainage channel comprising a main microfluidic channel and a set of secondary microfluidic channels connecting the main channel to the associated microfluidic accesses.

This makes it possible to homogenise and increase the density of access points for optimal drainage.

According to a first preferred embodiment of the present invention, said microfluidic actuating circuit is formed of a coil-shaped microfluidic actuating channel, called actuating coil, having a characteristic length L between two successive valves belonging to a same group of valves, a width w, a height h, and a deformation parameter A, bearing out the following double inequality:

$$1 \times {10}^{- 3} \leq A \frac{{wL}^{2}}{{h}^{3}} \leq 10$$

and preferably

$$0.01 \leq A \frac{{wL}^{2}}{{h}^{3}} \leq 1.$$

This makes it possible to create in a simple manner a transitory pressure gradient between every two successive valves of a same group of valves bringing about in an efficient manner a peristaltic pumping effect on each microfluidic drainage channel and whatever the length of the channel.

According to a second preferred embodiment of the present invention, said microfluidic actuating circuit comprises comb-shaped actuating channels defining capacitive elements, connected to a connection channel defining resistive elements, the valves being located on said actuating channels.

Advantageously, the downstream ends of said main microfluidic channels are connected to a same sink, the other ends being closed.

According to a variant, the set of main microfluidic channels is constituted of separate first and second sub-assemblies of main microfluidic channels, the first sub-assembly of main microfluidic channels being connected to a first sink and the second sub-assembly of main microfluidic channels being connected to a second sink.

Advantageously, said pressure generator is configured to generate pressure slots to actuate in a cyclical manner the valves belonging to each group of valves.

According to a third preferred embodiment of the present invention, said network of microfluidic channels comprises a main spiral-shaped microfluidic channel connected via secondary microfluidic channels to said set of microfluidic accesses, and the surface pump comprises at least three separate microfluidic actuating circuits suited to be connected to at least three separate pressure generators, each actuating circuit being constituted of a spiral-shaped actuating channel, said at least three actuating channels cooperating with each secondary microfluidic channel to form at least three valves configured to be actuated sequentially by said at least three pressure generators.

Advantageously, the surface pump comprises a first substrate, a deformable membrane, and a second substrate, said at least one actuating circuit being delimited by said deformable membrane and said first substrate, said network of microfluidic channels being delimited by said deformable membrane and said second substrate, said network of microfluidic channels and said at least one actuating circuit then being separated by said deformable membrane at the level of the intersecting zones thus forming a valve at each intersection.

Advantageously, the surface pump comprises a determined number of valves comprised between around 10 and 1000.

The invention also pertains to a pumping system comprising a surface pump according to any of the preceding characteristics, further comprising at least one pressure generator connected to said at least one actuating circuit of said surface pump.

The invention also relates to a dressing comprising a surface pump according to any of the preceding characteristics, comprising at least one sink connected to said surface pump, said at least one sink being intended to collect the fluid of interest pumped by the surface pump.

The invention also pertains to a dressing for exudative wounds comprising a set of microfluidic drainage channels provided with a plurality of microfluidic accesses defining a predetermined domain suited to covering a wound secreting exudate, a microfluidic actuating circuit suited to being connected to a single pressure generator, said microfluidic actuating circuit intersecting said microfluidic drainage channels on a plurality of deformable membrane intersecting zones forming a set of valves suited to causing, under an actuation of said pressure generator, a peristaltic effect pumping of the exudate circulating in said microfluidic drainage channels from said set of microfluidic accesses to at least one sink connected to said set of microfluidic drainage channels.

Thus, unlike the negative pressure technique, the drainage is incorporated in the dressing in a localised and non-global manner. Furthermore, it is not necessary to make the dressing leak-tight, which simplifies its application, its surveillance and its upkeep. Moreover, the dressing has a reduced bulk with a single connection path connected to a single external pressure generator for pumping the exudate secreted over the whole surface of the wound. Moreover, the connections to the dressing are not in contact with the exudates and thus the external pressure generator is not soiled by exudates and may be reused. Another advantage is the fact that the pressure generator used by the dressing is not bulky, is portable and employs very simple to implement technology.

According to a first preferred embodiment of the dressing, the microfluidic actuating circuit is formed of a coil-shaped microfluidic actuating channel.

According to a second preferred embodiment of the dressing, said microfluidic actuating circuit comprises comb-shaped actuating channels defining capacitive elements, connected to at least one connection channel defining a resistive element, the valves being located on said actuating channels.

Advantageously, said set of microfluidic drainage channels comprises a set of main microfluidic channels and a plurality of secondary microfluidic channels, each microfluidic drainage channel comprising a main microfluidic channel and a set of secondary microfluidic channels connecting the main channel to the associated microfluidic accesses.

Advantageously, the dressing comprises first and second sinks for collecting the exudates secreted by the wound and said set of main microfluidic channels is constituted of separate first and second sub-assemblies of main microfluidic channels, the first sub-assembly of main microfluidic channels being connected to the first sink and the second sub-assembly of main microfluidic channels being connected to the second sink.

Thus, the sinks are incorporated in the dressing even further facilitating application and eliminating the connections to an external waste receptacle.

Advantageously, the set of microfluidic drainage channels and the actuating fluidic channel are made of biocompatible material.

Thus, these channels may be deposited directly on the wound.

Advantageously, the dressing comprises a support layer receiving the set of microfluidic drainage channels and the actuating fluidic channel, said support layer being formed of a biocompatible porous material intended to be in contact with the wound.

Advantageously, the dressing comprises an external self-adhesive layer.

This makes it possible to protect the wound and to facilitate the application of the dressing on the wound.

Other advantages and characteristics of the invention will become clear from the detailed non-limiting description below.

### BRIEF DESCRIPTION OF THE DRAWINGS

Embodiments of the invention will now be described, as non-limiting examples, while referring to the appended drawings, in which:

FIG. 1 very schematically illustrates a surface pump, according to the invention;

FIG. 2 schematically illustrates a surface pump, according to a preferred embodiment of the invention;

FIG. 3 schematically illustrates a surface pump, according to a first preferred embodiment of the invention;

FIG. 4 is an experimental example of a pumping system illustrating the action of a single microfluidic actuating circuit on a microfluidic channel, according to the embodiment of FIG. 3;

FIG. 5 schematically illustrates a surface pump, according to a second preferred embodiment of the invention;

FIG. 6 schematically illustrates a surface pump, according to a third preferred embodiment of the invention;

FIGS. 7A-7F schematically illustrate a method of manufacturing a surface pump, according to the invention;

FIG. 8 very schematically illustrates a pumping system according to an embodiment of the invention;

FIG. 9 schematically illustrates a dressing comprising a surface pump, according to a preferred embodiment of the invention; and

FIGS. 10A and 10B schematically illustrate a dressing according to first and second preferred embodiments of the invention.

### DETAILED DESCRIPTION OF A PREFERRED EMBODIMENT

The basic concept of the invention consists in producing a surface drainage system from a network of peristaltic micropumps and applying it to a dressing for exudative wounds.

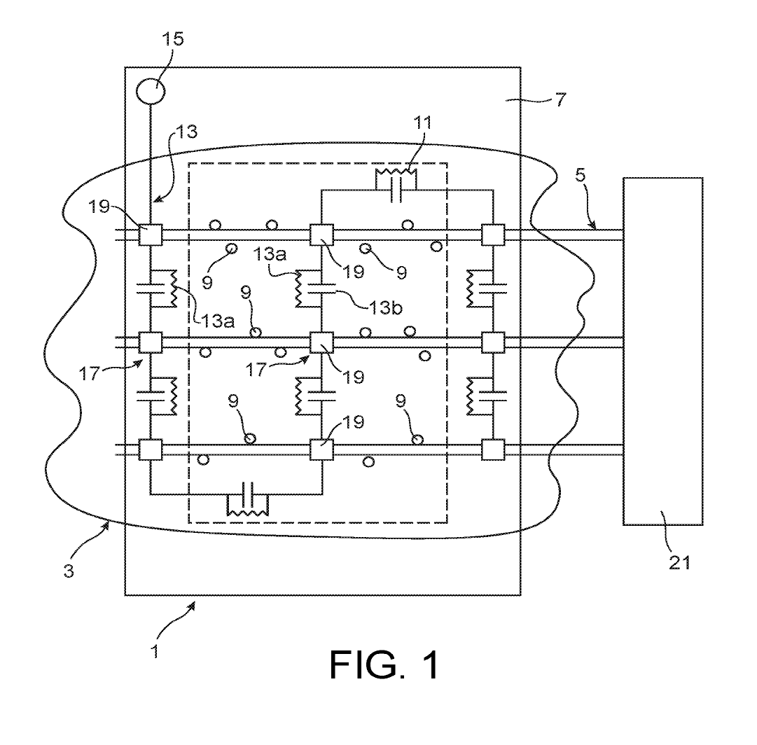

FIG. 1 very schematically illustrates a surface pump, according to the invention.

The surface pump 1 comprises a network 3 of microfluidic channels 5 transporting a liquid of interest to drain, and pumping means 7 to actuate the pumping in these microfluidic channels.

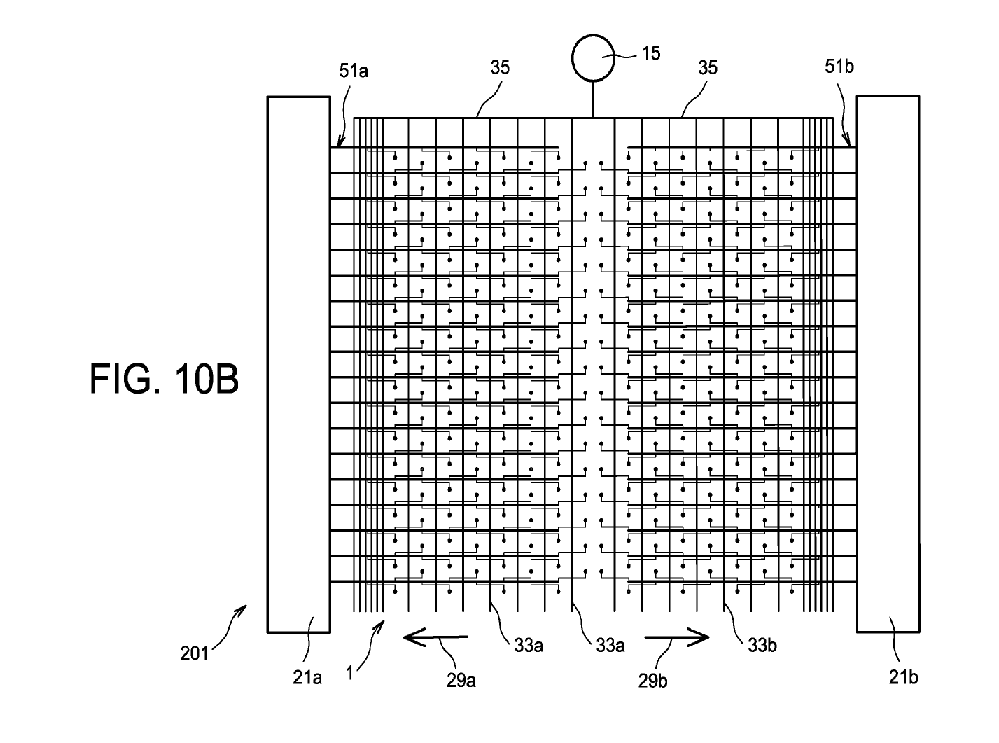

The network 3 of microfluidic channels 5 includes a set of microfluidic accesses 9 arranged in a predetermined domain 11 suited to covering a surface source of the fluid of interest. The microfluidic accesses 9 may be distributed in a homogeneous and planar manner in the predetermined domain 11. The border of the predetermined domain 11 may be any geometric shape (for example, rectangular, circular, oval) which adapts itself to the shape of the surface source of the fluid. As an example, the microfluidic accesses 9 are arranged in a matrix fashion (see for example FIG. 10A).

The pumping means 7 comprise at least one microfluidic actuating circuit 13 represented very schematically in FIG. 1 by a rheological model. More particularly, the fluidic actuating elements constituting each microfluidic actuating circuit are modelled by resistive 13a and capacitive 13b fluidic elements defined by resistance and capacity fluidic quantities.

Each actuating circuit 13 is suited to being connected to a pressure generator 15 and contains a fluid of determined viscosity, selected from a set of fluids comprising for example, water, air, oil, or other types of fluids according to the application.

The fluidic actuating elements are geometrically and materially configured to enable each actuating circuit 13 to intersect the network 3 of microfluidic channels 5 on a plurality of nodes or deformable membrane intersecting zones 17 forming a network or set of valves 19 spread out within the network 3 of microfluidic channels. Each valve 19 is formed by the intersection (i.e. the superposition) of a fluidic actuating element with a corresponding microfluidic channel 5. Thus, a valve 19 belonging to a microfluidic channel 5 is suited to compressing and closing this channel, partially or totally under the pressurisation of the fluidic actuating element cooperating with the microfluidic channel.

The set of valves 19 is suited to causing under the controlled actuation of the pressure generator(s) 15 a pumping of the fluid of interest circulating in the network 3 of microfluidic channels. In fact, the membrane at the level of each valve 19 applies a mechanical action to the fluid in the corresponding microfluidic channel, causing the displacement of the fluid in this channel. The successive actuation of valves 19 belonging to a same microfluidic channel 5 generates a so-called peristaltic micropumping effect. In other words, the group of valves 19 belonging to each microfluidic channel 5 forms a set of peristaltic micropumps coordinated together to create all along the channel a microperistaltic pumping of the fluid which introduces itself into the microfluidic channel via the different microfluidic accesses 9. Thus, the network 3 of microfluidic channels 5 forms a network of micropumps.

The document FR2974598 describes an example of a peristaltic micropump made of silicon comprising three deformable membranes actuated by three separate piezoelectric elements. Other examples are described in the publication “Monolithic Microfabricated Valves and Pumps by Multilayer Soft Lithography”, Marc Unger et al. Science 288, 113 (2000). These examples concern in general linear micropumps comprising a single microfluidic channel transporting the liquid and three pneumatic channels controlled sequentially by three actuators.

Nevertheless, according to an aspect of the invention, the surface pump 1 is constituted of a network of peristaltic micropumps draining a fluid of interest on several flowing paths from a set or a matrix of microfluidic accesses 9 which can cover a large surface.

Thus, the valves 19 control the circulation of the fluid of interest in the network 3 of microfluidic channels 5 to cause a peristaltic effect pumping of the fluid from the set of microfluidic accesses 9 to at least one sink 21 connected to the network 3 of microfluidic channels 5.

The surface pump according to the invention may be used in numerous fluidic systems such as, as examples, labs-on-chips, or instead hydraulic circuits for cooling electronic chips.

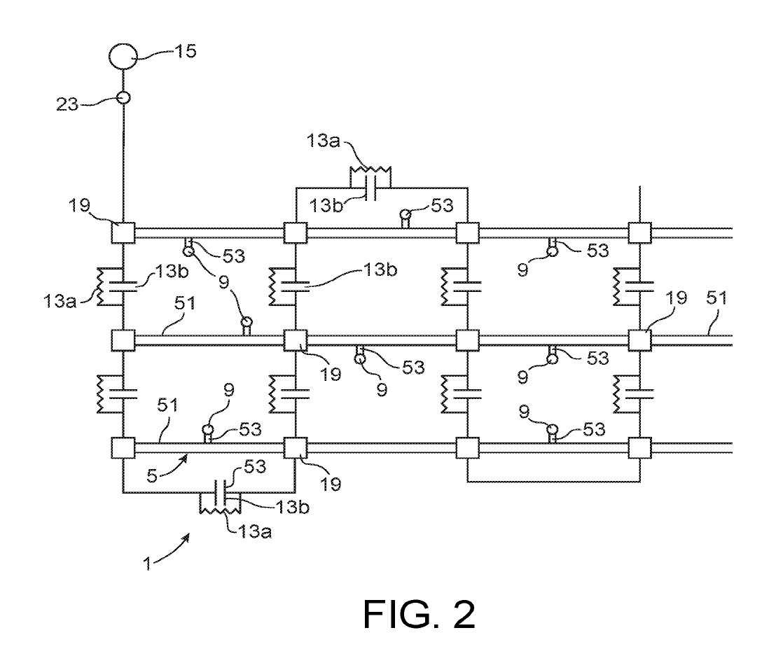

FIG. 2 schematically illustrates a surface pump, according to a preferred embodiment of the invention.

According to this embodiment, the network of microfluidic channels comprises a set of separate microfluidic drainage channels 5 provided with a set of microfluidic accesses 9.

The set of microfluidic drainage channels 5 may for example comprise a set of main microfluidic channels 51 and a plurality of secondary microfluidic channels 53. In this case, each microfluidic drainage channel 5 comprises a main microfluidic channel 51 and a set of secondary microfluidic channels 53 connecting the main channel 51 to the associated microfluidic accesses 9.

Furthermore, each microfluidic drainage channel 5 is associated with a determined group of valves with at least one valve 19 (and preferably several valves) per secondary channel 53. The drawing shows three microfluidic drainage channels 5 with a group of four valves 19 per main channel 51 arranged such that there is one valve per secondary channel 53, but obviously the invention may apply to a surface pump comprising any number of microfluidic drainage channels 5 as well as any number of valves 19 per group and, in addition, different groups may comprise different numbers of valves.

An advantageous particularity of this embodiment is that the surface pump 1 comprises a single microfluidic actuating circuit 13 suited to being connected to a single pressure generator 15 via a single connection port (or inlet) 23. This actuating circuit 13 is constituted of resistive 13a and capacitive 13b fluidic actuating elements defining a response time delay between two successive valves 19 belonging to a same group of valves (i.e. on a same microfluidic drainage channel 51). The response time delay is created by the fact that the resistive and capacitive fluidic actuating elements generate a transitory pressure gradient between each two successive valves 19 of a microfluidic drainage channel 51.

The response time delay is configured to cause, under an action of the pressure generator 15, a sequential actuation of the valves 19 belonging to each group of valves then creating a peristaltic pumping in the microfluidic drainage channel 51.

Moreover, the pressure generator 15 is configured to generate pressure slots making it possible to actuate the valves 19 belonging to each group of valves 19 in a cyclical manner. It will be noted that a cycle corresponds to the propagation front of the opening then the closing of the valves 19. Thus, when a high pressure is applied to the microfluidic actuating circuit 13, the valves 19 of each group of valves pass firstly from an open state to a closed state one after the other thanks to the response time delay and then, when a low pressure is applied to the fluidic circuit, the valves 19 of each group of valves pass from the closed state to an open state one after the other.

The actuating response time delay between two successive valves 19 Vₙ₋₁, Vₙ of a microfluidic drainage channel 51 is determined by the resistance and capacity values of the different fluidic elements 13a, 13b between these two valves. It will be noted that for monophasic flows, the electrical equivalence of a microfluidic circuit is possible thanks to a linearization of the Stokes equation.

Thus, the response time delay may be expressed by a time constant τ defined by the product τ=R×C between the fluidic resistance R and the fluidic capacity C relative to the two successive valves 19.

The notion of fluidic resistance R is linked to the dissipation of energy and may be expressed as the ratio between the head loss ΔP and the flow rate Q through a microfluidic channel.

The fluidic capacity C may be defined by the volumetric expansion δV of a microfluidic channel following a pressure increase δP.

Advantageously, the fluidic resistance and capacity values are chosen so that the response time delay τ is comprised between a millisecond and a minute and preferably, between around 0.01 second and several seconds.

The fluidic resistance and capacity values are determined by the material dimensioning and the composition of the actuating circuit 13.

In fact, the fluidic resistance R of a microfluidic channel is a function of its characteristic length L, its width w, its height h, a parameter K depending on the shape of its section, as well as the viscosity μ of the fluid contained in the channel. More particularly, the fluidic resistance R is defined by the following expression:

$$R = \frac{K μ L}{{wh}^{3}} \qquad ( 1 )$$

the parameter K is in general comprised between 12 and 32 and in particular, K=32 for a channel of square section and K=12 for a channel of rectangular section with h<w.

The fluidic capacity C may be expressed as a function of the dimensions of the channel, its Young's modulus E and a parameter a which depends on the material constituting the channel, according to the following expression:

$$C = α \frac{{Lw}^{2}}{E} \qquad ( 2 )$$

In particular, the equations (1) and (2) indicate that by increasing the width w of a channel, its fluidic resistance R is decreased while its capacity C is increased and vice-versa. Thus, it is possible for example to increase the width w of the channels at the levels of the intersections to increase the expansion and the efficiency of the valves.

Thus, each section of actuating channel between two intersections may be assimilated to an elementary circuit RᵢCᵢ in parallel. This explains why in applying a pressure variation at the inlet 23 of the actuating channel 13, this pressure variation propagates between two successive valves of the actuating circuit 13 according to a response time τᵢ=Rᵢ×Cᵢ. Generally speaking, the actuating circuit comprises a series of valves V₁, . . . , Vₙ, . . . , V_{N}. Each valve Vₙ is characterised by a response time τₙ=Rₙ×Cₙ, Rₙ and Cₙ characterising the actuating circuit between the valve Vₙ and the inlet 23 of the actuating circuit 13. The greater the distance between a valve Vₙ and the inlet 23 of the circuit 13, the greater the response time τₙ.

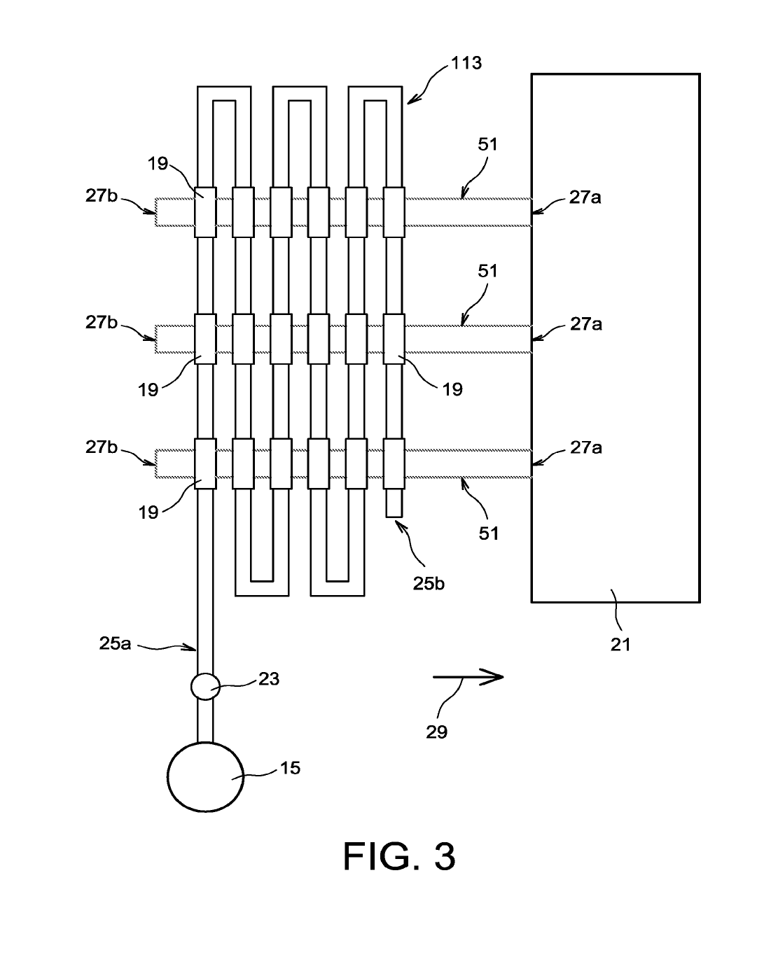

FIG. 3 schematically illustrates a surface pump, according to a first preferred embodiment of the invention. In the interest of simplification, the microfluidic accesses are not illustrated in the drawing of FIG. 3.

According to this first embodiment, the microfluidic actuating circuit is formed of a coil-shaped actuating microfluidic channel 113, called actuating coil 113.

The example illustrated in FIG. 3 shows that an end 25a of the actuating coil 113 is connected via the single connection port 23 to the pressure generator 15, whereas the other end 25b is closed. Furthermore, the downstream ends 27a with respect to the pumping direction (indicated by the arrow 29) of the main microfluidic channels 51 are connected to a same sink 21, the other ends 27b being closed.

According to a variant (see the example of FIG. 10A), the set of main microfluidic channels 51 is constituted of separate first and second sub-assemblies of main microfluidic channels 51a, 51b. The first sub-assembly of main microfluidic channels 51a is connected to a first sink 21a and the second sub-assembly of main microfluidic channels 51b is connected to a second sink 21b. In this case, the ends of the actuating coil 25a, 25b are closed and an intermediate point 25c between these two ends is connected to the pressure generator 15 (FIG. 10A).

According to one or the other variant, the actuating coil 113 has a characteristic length L between for example two successive valves 19 belonging to a same group of valves, a width w, and a height h.

Using the above equations (1) and (2), the response time delay τ is given by the following expression:

$$τ = RC = \frac{K μ L}{{wh}^{3}} \times \frac{α {Lw}^{2}}{E} = A \frac{{wL}^{2}}{{h}^{3}} \qquad ( 3 )$$

where

$$A = \frac{K αμ}{E}$$

is a deformation coefficient or parameter which depends on the Young's modulus E of the material of the actuating coil 113 and the viscosity coefficient μ of the fluid contained in the actuating coil 113.

Advantageously, the material and dimensions of the actuating coil 113 are selected so as to bear out the following double inequality:

$$1 \times {10}^{- 3} \leq A \frac{{wL}^{2}}{{h}^{3}} \leq 10 ,$$

and preferably

$$0.01 \leq A \frac{{wL}^{2}}{{h}^{3}} \leq 1.$$

As an example, for a surface pump made of PDMS composed of a mixture of ten volumes of polymer for one volume of cross-linking agent, the Young's modulus is E=2.5×10⁶ Pa, the parameter a is practically equal to 0.5 and for an actuating coil 113 of rectangular section filled with water, the viscosity is μ=0.001 Pa·s and K is a little greater than 12. In this case, the deformation parameter A is of the order of 3×10⁻⁹. For example with an actuating coil 113 of width w=65 μm and height h=10 μm, it suffices to have a characteristic length L between two successive valves 19 of the order of 3 cm to target a response time τ of the order of 0.1 s.

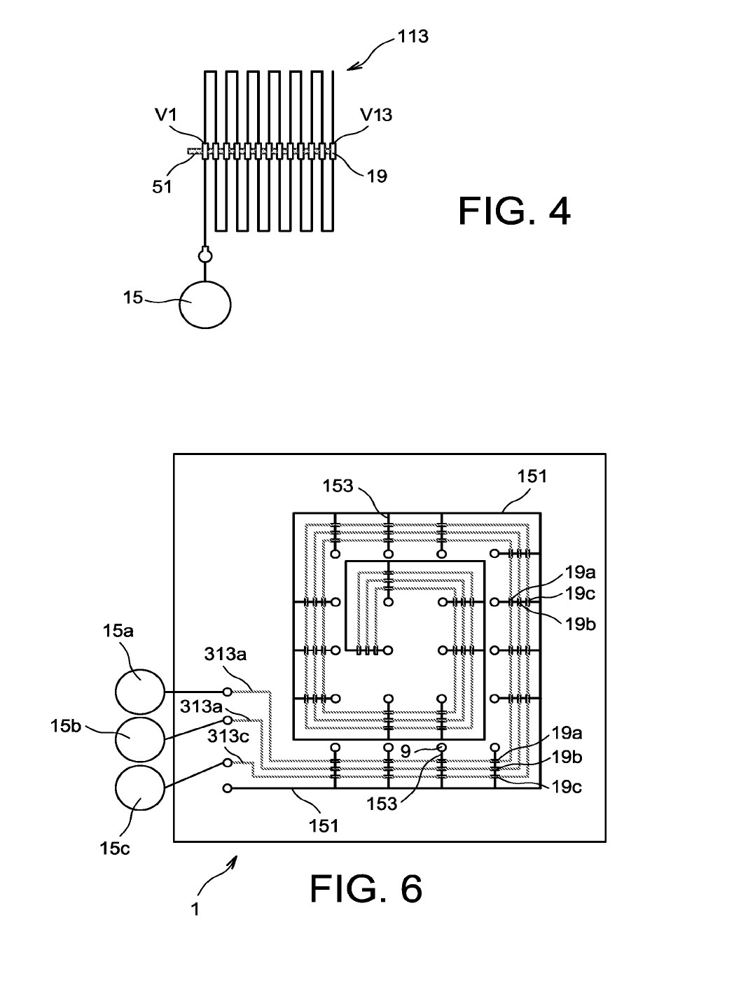

FIG. 4 is an experimental example of a pumping system illustrating the action of a single microfluidic actuating circuit on a microfluidic channel, according to the embodiment of FIG. 3.

According to this illustrative example, the actuating coil 113 intersects the microfluidic drainage channel to form a series of 13 valves V₁, . . . , V₁₃ with a pitch of 400 μm. The actuating coil 113 has a total length between the valves V₁ and V₁₃ of 90 mm, a width w of 65 μm, and a height h=10 μm (according to a first version of the pumping system) and a height h=50 μm (according to a second version of the pumping system). The width of the coil at the level of the valves is 200 μm. The microfluidic drainage channel has a width of 200 μm and the total surface area of the pumping system is around 6 mm×6 mm.

A pressure generator 15 generating pressure slots of amplitude P₋=−100 mbar to P₊=600 mbar at a determined frequency is connected to the actuating coil 113 of the pumping system. It will be noted that a cycle corresponds to the propagation front of the opening then the closing of the valves.

According to the first version (h=10 μm), the frequency of the pressure generator is chosen equal to 0.1 Hz. The measured flow rate is of the order of 0.8 nl per cycle i.e. around 4 nl/mn. The total response time delay observed between the first valve V₁ and the last valve V₁₃ is between 1.2 s and 4.4 s. The value of 1.2 s is observed when a positive pressure is applied, which leads to the closing of successive valves, whereas the value of 4.4 s is measured when a negative pressure is applied, which leads to the opening of successive valves. This difference results from non-linear effects not taken into account in the model of the present application. It will be noted that the theoretical model for the first version (h=10 μm) predicts a total response time delay between the first valve V₁ and the last valve V₁₃ of 1.3 s.

According to the second version (h=50 μm), the frequency of the pressure generator is comprised between 0.5 Hz and 4 Hz. The flow rate measured at a frequency of 1 Hz is of the order of 0.6 μl/mn i.e. around 10 nl per cycle. The measured flow rate is of the order of 1 μl/mn at a frequency of 2 Hz. The total response time delay observed between the first valve V₁ and the final valve V₁₃ is of the order of 0.13 s. It will be noted that the theoretical model for the second version of the pumping system predicts a total response time delay between the first valve V₁ and the final valve V₁₃ of 0.03 s.

The experimental values of the first and second versions show good correlation (less than a decade) between the theoretical model and experiment, considering quite simple modelling hypotheses.

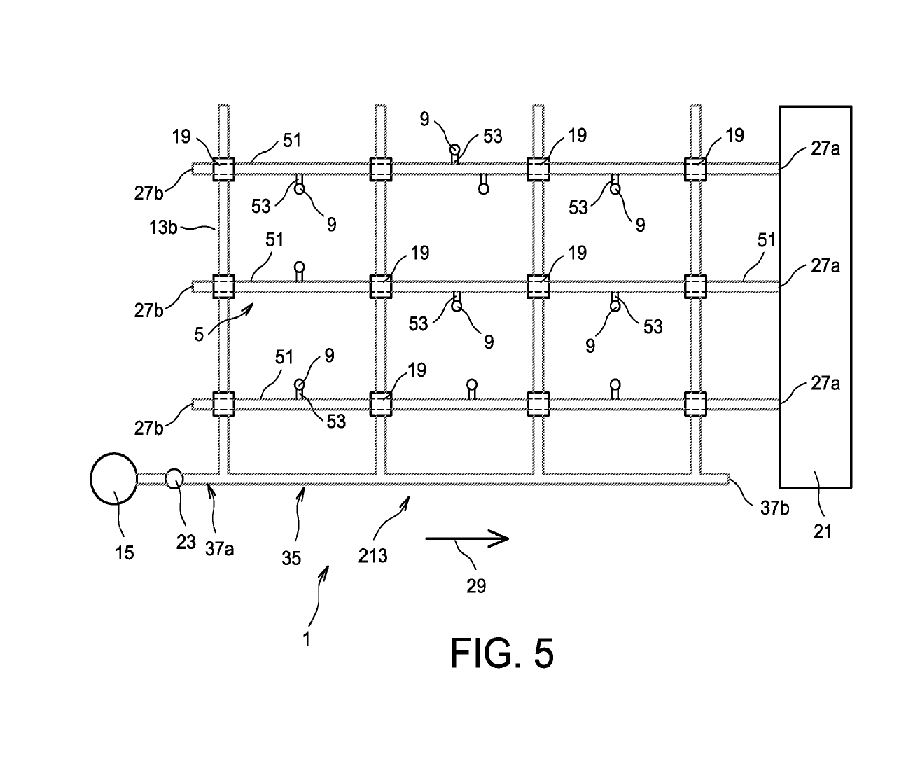

FIG. 5 schematically illustrates a surface pump, according to a second preferred embodiment of the invention.

In a similar manner to the preceding embodiment, the network of microfluidic channels comprises a set of separate microfluidic drainage channels 5 provided with a set of microfluidic accesses 9. The set of microfluidic drainage channels 5 comprises a set of main microfluidic channels 51 and a plurality of secondary microfluidic channels 53. Each microfluidic drainage channel 5 comprises a main microfluidic channel 51 and a set of secondary microfluidic channels 53 connecting the main channel to the associated microfluidic accesses 9.

Moreover, each microfluidic drainage channel 5 is associated with a determined group of valves. The drawing shows three microfluidic drainage channels 5 with a group of four valves 19 per channel.

Furthermore, the surface pump 1 comprises a single microfluidic actuating circuit 213. Nevertheless, according to this second embodiment, the single microfluidic actuating circuit 213 comprises comb-shaped actuating channels 33 mainly forming capacitive elements. The valves 19 are located on the actuating channels 33 and the latter are connected to a connection channel 35 which mainly forms a resistive element. The actuating channels 33 are for example substantially perpendicular to the connection channel 35. The fact that the actuating channels 33 have very low fluidic resistances in view of the fluidic resistance of the connection channel 35 makes it possible to create an actuating response time delay between two successive valves 19 belonging to a same group of valves, that is to say arranged along a same main microfluidic channel. More precisely, the response time delay depends as indicated previously on the expansion capacity of each actuating channel 33 and the fluidic resistance when flow is established for a given pressure in the actuating channel.

An end 37a of the connection channel 35 may be connected via a single connection port 23 to a single pressure generator 15. The other end 37b of the connection channel 35 as well as the free ends 37c of the actuating channels 33 are closed.

The response time delay is configured to cause, under an action of the pressure generator 15, a sequential actuation of the valves 19 belonging to each group of valves then creating a peristaltic pumping. Moreover, the pressure generator 15 is configured to generate pressure slots making it possible to actuate the valves belonging to each group of valves in a cyclical manner.

Advantageously, the downstream ends 27a (with respect to the pumping direction 29) of the main microfluidic channels 51 are connected to a same sink 21 whereas the other ends 27b are closed. According to this variant, the valves of each main microfluidic channel are better synchronised. It is considered that two adjacent valves, situated on two neighbouring channels, will be simultaneously activated.

According to a variant (see the example of FIG. 10B), the set of main microfluidic channels 51 is constituted of separate first and second sub-assemblies of main microfluidic channels 51a, 51b. The first sub-assembly of main microfluidic channels 51a is connected to a first sink 21a and the second sub-assembly of main microfluidic channels 51b is connected to a second sink 21b. In this case, two actuating channels 33a, 33b situated around the middle of the actuating circuit 213 are connected to a same pressure generator 15.

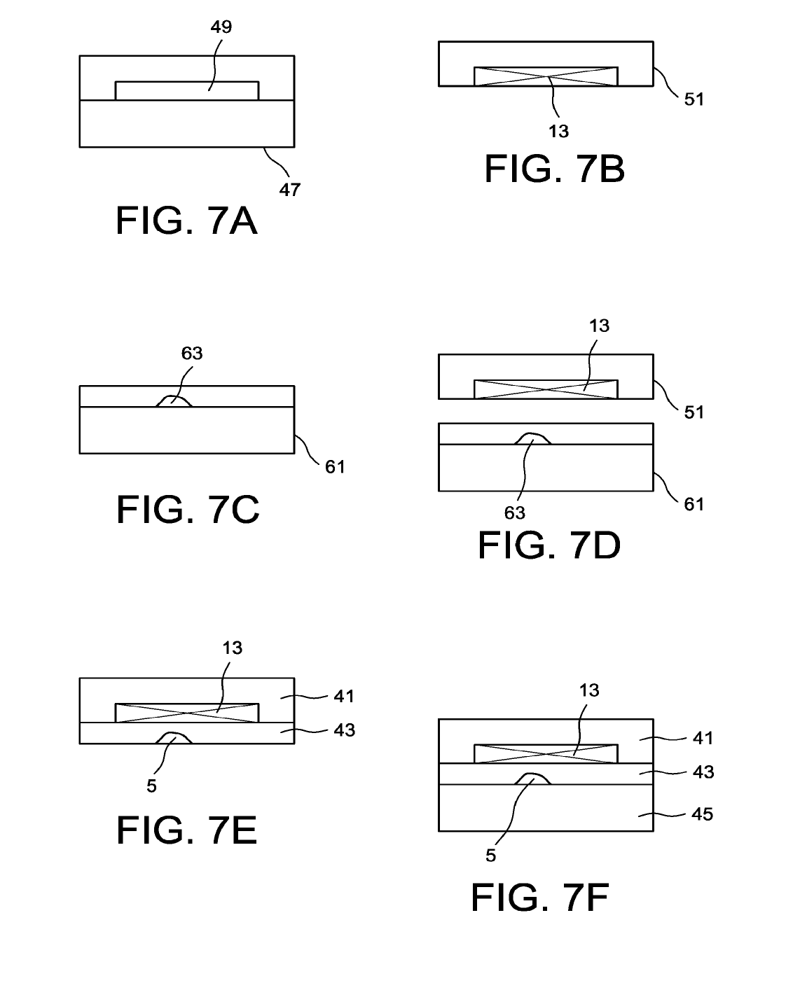

FIG. 6 schematically illustrates a surface pump, according to a third preferred embodiment of the invention.

According to this embodiment, the network of microfluidic channels comprises a single main spiral-shaped microfluidic channel 151, connected via secondary microfluidic channels 153 to a set of microfluidic accesses 9.

Moreover, the surface pump 1 comprises at least three separate microfluidic actuating circuits 313a, 313b, 313c suited to be connected to at least three separate pressure generators 15a, 15b, 15c. Each actuating circuit 313a, 313b, 313c is constituted of a spiral-shaped actuating channel similar to that of the main microfluidic channel 151. Thus, the actuating circuits 313a, 313b, 313c adjoin the main microfluidic channel 151 and cooperate with each secondary drainage channel 153 to form a grouping of at least three valves 19a, 19b, 19c. The valves belonging to each grouping of valves 19a, 19b, 19c are configured to be actuated sequentially by the pressure generators 15a, 15b, 15c.

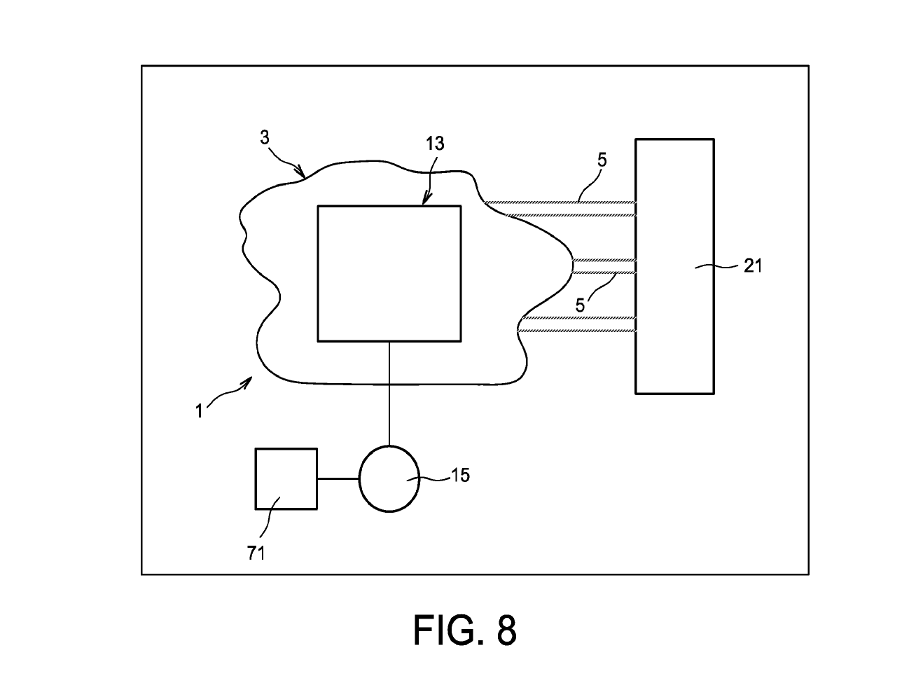

FIGS. 7A-7F schematically illustrate a method of manufacturing a surface pump, according to the invention.

The surface pump according to the different embodiments of the invention comprises a first substrate 41, a deformable membrane 43, and a second substrate 45 (see FIG. 7F). The actuating circuit(s) 13 is(are) delimited by the deformable membrane 43 and the first substrate 41, whereas the network of microfluidic channels 5 is delimited by the deformable membrane 43 and the second substrate 45. Thus, the network of microfluidic channels 5 and the actuating circuit(s) 13 are separated by the deformable membrane 43 at the levels of the intersecting zones forming a valve at each intersection.

The material used for the manufacture of the surface pump is a polymer of PDMS (polydimethylsiloxane) type according to for example a mixture of ten volumes of polymer for one volume of cross-linking agent. The manufacturing technique may be similar to that described for example in the document “Monolithic Microfabricated Valves and Pumps by Multilayer Soft Lithography”, Marc Unger, et al. Science 288, 113 (2000).

More particularly, FIG. 7A shows a step of manufacturing the first substrate 51 by the moulding of the polymer on an impression 47 comprising a pattern 49 intended to form the future microfluidic actuating circuit 13.

After polymerisation, the first substrate 51 is removed from the impression mould 47 as illustrated in FIG. 7B.

FIG. 7C shows the production of the membrane by polymerisation of a thin layer of PDMS spread by spin coating on an impression 61 comprising a pattern 63 intended to form the future network of microfluidic channels 5. As an example, the thickness of the membrane is around 15 μm.

After polymerisation (FIG. 7D), the first substrate 51 is assembled to the membrane 43 and they are bonded together, for example by O₂ plasma as illustrated in FIG. 7D.

FIG. 7E shows that the bi-layer formed by the first substrate 51 and the membrane 43 is removed from the impression mould 43, then assembled and bonded onto a second substrate made of PDMS for example, by O₂ plasma as illustrated in FIG. 7F.

Holes (not represented) are formed through the substrate 45 by punching before the assembly and bonding step.

Moreover, the present invention relates to a pumping system comprising a surface pump according to any of the preceding embodiments.

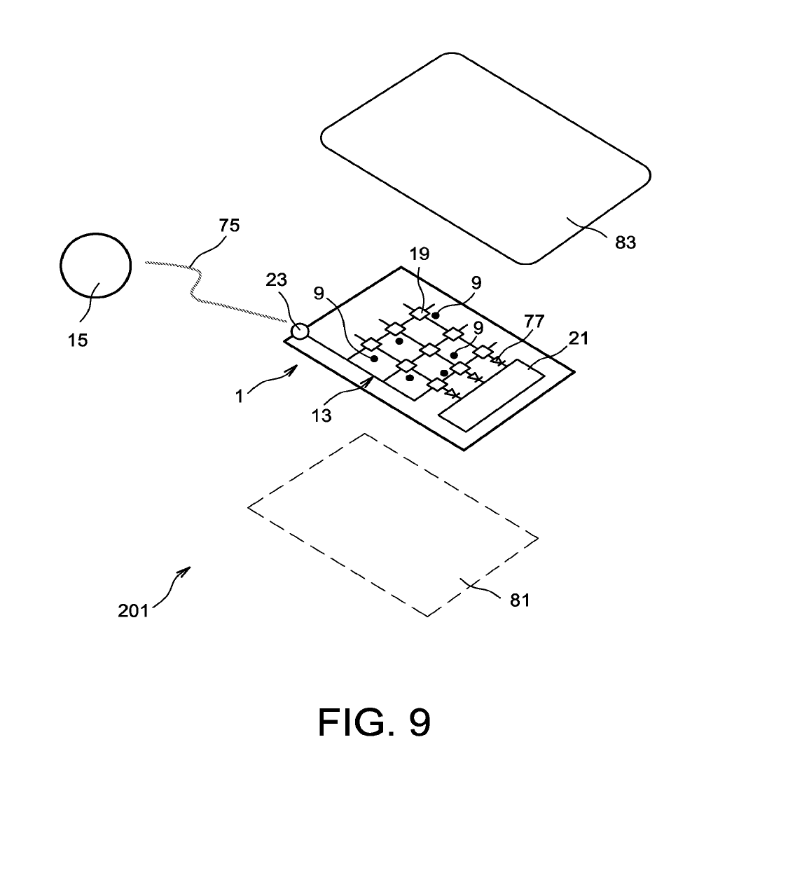

FIG. 8 very schematically illustrates a pumping system according to an embodiment of the invention.

The pumping system 101 comprises at least one pressure generator 15 connected to the actuating circuit(s) 13 of the surface pump 1 as well as a control device 71 of the pressure generator 15. The control device 71 comprises for example an electrical supply unit and a processing unit intended to control the pressure generator 15.

Advantageously, the pumping system 101 comprises at least one sink 21 connected to the network 3 of microfluidic channels 5 of the surface pump.

The present invention also relates to a dressing incorporating a surface pump according to any of the preceding claims. The dressing is made of a flexible and biocompatible material and comprises at least one sink connected to the network of microfluidic channels. The sink is intended to collect the fluid of interest pumped by the surface pump.

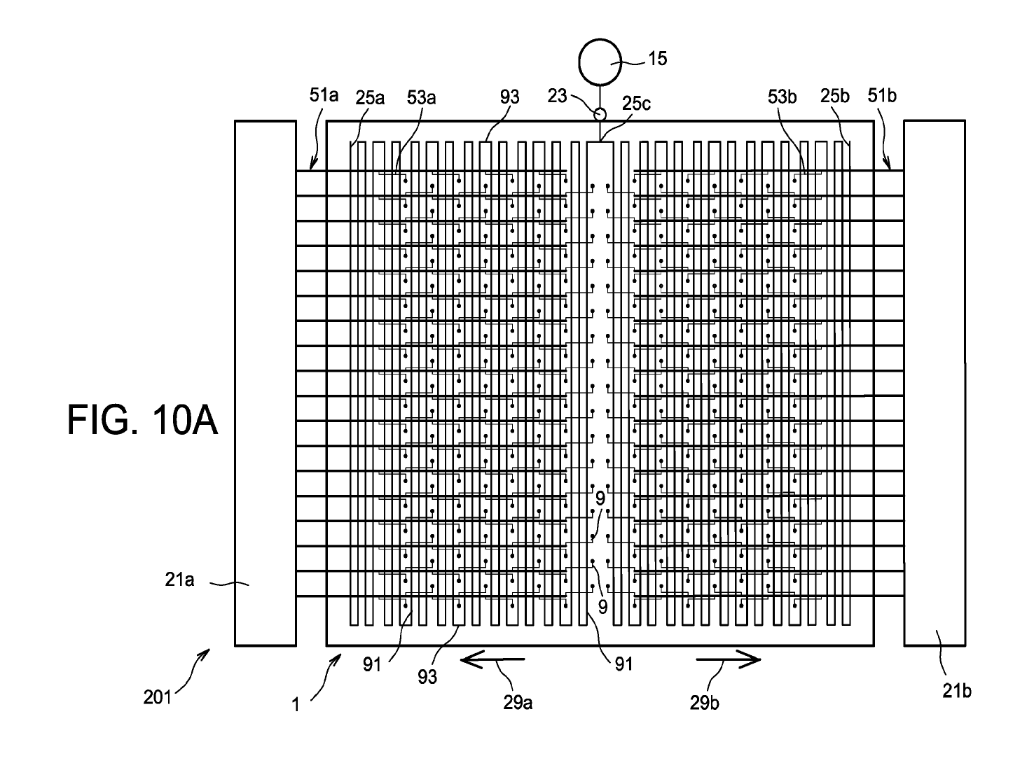

FIG. 9 schematically illustrates a dressing for exudative wounds comprising a surface pump, according to a preferred embodiment of the invention.

According to this embodiment, the dressing 201 comprises a set of microfluidic drainage channels 5 and a microfluidic actuating circuit 13.

The set of microfluidic drainage channels 5 is provided with a plurality of localised microfluidic accesses 9 defining a predetermined domain suited to covering a wound secreting exudate.

The microfluidic actuating circuit 13 comprises a single connection port 23 making it possible to reduce the bulk of the dressing. The latter makes it possible to connect in a detachable manner the microfluidic actuating circuit 13 via for example a connection 75 to a single pressure generator 15. The fact that the microfluidic actuating circuit 13 is directly connected to the generator 15 avoids resorting to a leak-tight dressing 201, which simplifies its application, its surveillance, and its upkeep.

It will be noted that a single pressure generator 15, not bulky and portable, suffices to carry out the drainage according to the invention. In addition, the pressure generator 15 is never in contact with the exudates and may thus be reused in complete security.

The microfluidic actuating circuit 13 is produced so as to intersect the microfluidic drainage channels 5 on a plurality of deformable membrane intersecting zones forming a network of valves 19.

The valves 19 are suited to causing under a controlled actuation of the pressure generator 15 a microperistaltic effect pumping of the exudate circulating in the microfluidic drainage channels 5 from the set of microfluidic accesses 9 to at least one sink 21 connected to the set of microfluidic drainage channels. Thus, the drainage of the exudate is carried out efficiently in an independent manner on each zone among a multitude of zones and independently of the fact that the exudation rate can vary between the different zones.

Advantageously, said at least one sink 21 is incorporated in the dressing 201. This facilitates the application of the dressing and eliminates connections to an external waste receptacle. The sink 21 may be made of a very hydrophilic and very absorbent material.

Advantageously, the set of microfluidic drainage channels 5 and the microfluidic actuating circuit 13 are made of flexible and biocompatible material making it possible to deposit them directly on the wound or close to the latter.

In a variant, the dressing may comprise a support layer 81 on which are arranged the surface pump 1 and the sink(s) 21. The support layer 81 is made of a porous biocompatible material (for example, made of fabric) intended to be in contact with the wound.

Moreover, the dressing 201 comprises an external protective layer 83 for example, in the form of a self-adhesive film to fix the dressing 201 onto the wound and to protect the wound, the surface pump 1, and the sink(s).

FIG. 10A schematically illustrates a dressing according to a first preferred embodiment of the invention.

According to this first embodiment, the microfluidic accesses 9 are arranged in a regular manner line by line and column by column forming a matrix of microfluidic accesses 9 with m lines and n columns. The position (i, j) of each microfluidic access 9 may be defined by the line number i and the column number j, with i=1, . . . , m and j=1, . . . , n. The matrix of microfluidic accesses occupies a rectangular surface intended to cover the wound.

Furthermore, the set of micro-fluidic drainage channels comprises a set of main microfluidic channels 51 substantially parallel with each other as well as a plurality of secondary microfluidic channels connecting the microfluidic accesses 9 to the main channels 51.

The main microfluidic channels 51 are arranged along lines substantially parallel to the lines of the matrix of microfluidic accesses. More particularly, each line of microfluidic accesses 9 is framed by two main microfluidic channels 51. Thus, the dressing 201 may comprise n+1 lines of main microfluidic channels 51 such that each line of microfluidic accesses 9 is situated between two consecutive channels 51.

Advantageously, the microfluidic accesses 9 arranged on a line are connected in an alternate manner via secondary microfluidic channels 53, with the two main framing microfluidic channels 51. For example, the microfluidic accesses 9 are connected with one or the other of the main framing microfluidic channels 51, according to the parity of their column indices.

Advantageously, the set of microfluidic accesses 9 forms a bijective relation with the set of secondary microfluidic channels 53. In other words, each microfluidic access 9 is connected to a main fluidic channel 51 via one and a single secondary fluidic channel 53.

According to this embodiment, the set of channels 51 is constituted of two separate sub-assemblies of main microfluidic channels 51a, 51b. A first sub-assembly of main channels 51a is associated with the first half of the columns (j=1, . . . , n/2) of the matrix of microfluidic accesses 9 and a second sub-assembly of main channels 51b is associated with the second half of the columns (j=n/2+1, . . . , n) of the matrix of microfluidic accesses 9.

The first sub-assembly of main microfluidic channels 51a is connected to a first sink 21a. More particularly, the first sink 51a is connected to the ends of the main microfluidic channels 51a which are on the side of the first column (j=1) of microfluidic accesses 9. Similarly, the second sub-assembly of main microfluidic channels 51b is connected to a second sink 21b. The latter is connected to the ends of the main microfluidic channels 51b which are on the side of the final column (j=n) of microfluidic accesses 9. The other ends of the main microfluidic channels 51a, 51b of the first and second sub-assemblies are closed.

As an example, the main microfluidic channel 51a situated between the lines i and i+1 and belonging to the first sub-assembly of channels is connected in an alternating manner to the microfluidic accesses 9 of indices (i, 2k−1) and (i+1, 2k) with k=1, . . . , n/4. Similarly, the main microfluidic channel 51b situated between the lines i and i+1 and belonging to the second sub-assembly is connected in an alternating manner to the microfluidic accesses 9 of indices (i, 2k−1) and (i+1, 2k) with k=n/4+1, . . . , n/2.

Furthermore, according to this first embodiment, the microfluidic actuating circuit is formed of a coil-shaped microfluidic actuating channel 113. The two ends 25a, 25b of the actuating coil 113 are closed whereas an intermediate point 25c between these two ends is connected via a single connection port 23 to a pressure generator 15.

The actuating coil 113 is configured to intersect each main microfluidic channel 51a, 51b as well as each secondary microfluidic channel 53a, 53b at least once between each two consecutive microfluidic accesses 9 situated on a same line.

More particularly, the actuating coil 113 is a periodic slot-shaped microfluidic channel with vertical actuating segments 91 (substantially perpendicular to the lines of microfluidic accesses 9) and horizontal segments 93. At least one vertical actuating segment 91 is arranged between two consecutive columns of microfluidic accesses 9. Moreover, the two vertical actuating segments 91 of the middle intersecting respectively the first and second sub-assemblies of microfluidic channels 51a, 51b, are connected to the pressure generator 51.

Advantageously, as in the example of FIG. 10A, three vertical actuating segments 91 or more are arranged between each two consecutive columns of microfluidic accesses 9. Thus, each microfluidic access 9 is associated with at least three valves such that the exudate penetrating into each microfluidic access is pumped in a peristaltic manner to the corresponding sink 21a, 21b without any risk of backflow. The valves are arranged at the intersecting zones between the vertical actuating segments 91 and the main 51a, 51b and secondary 53a, 53b microfluidic channels and not represented in FIG. 10A in the interest of simplification. The arrows 29a and 29b indicate the direction of opening/closing propagation of the valves inducing the drainage of the exudate to the lateral sinks 21a and 21b.

FIG. 10B schematically illustrates a dressing according to a second preferred embodiment of the invention.

This second embodiment differs from that of FIG. 10A uniquely by the fact that the microfluidic actuating circuit comprises a set of comb-shaped actuating channels substantially perpendicular to the lines of the microfluidic accesses, and defining capacitive elements. This set of actuating channels is constituted of two groups of actuating channels. A first group of actuating channels 33a is associated with the first sub-assembly of main channels 51a and a second group of actuating channels 33b is associated with the second sub-assembly of main channels 51b.

The first and second groups of comb-shaped actuating channels 33a are connected to a connection channel 35 substantially parallel to the microfluidic access lines. In variants, the first and second groups of actuating channels 33a, 33b may be connected respectively to separate first and second connection channels 35 (not represented). The valves are located on the first and second groups of actuating channels 33a, 33b.

As in the preceding example, at least one vertical actuating channel 33a, 33b is arranged between each two consecutive columns of microfluidic accesses 9 and the two vertical actuating channels of the middle intersecting respectively the first and second sub-assemblies of microfluidic channels are connected to the pressure generator 15.

Advantageously, three vertical actuating channels 33a or more are arranged between each two consecutive columns of microfluidic accesses 9 such that each microfluidic access is associated with at least three valves.

Finally, the dressing according to the invention may comprise a surface pump according to other embodiments such as for example that of FIG. 6.

1. Surface pump comprising a network (3) of microfluidic channels (5) and pumping means (7), characterised in that said network of microfluidic channels includes:
a set of microfluidic accesses (9) distributed in a predetermined domain (11) adapted to covering a surface source of a fluid of interest,
and in that said pumping means comprise at least one microfluidic actuating circuit (11) intersecting said network of microfluidic channels on a plurality of deformable membrane intersecting zones (17) forming a set of valves (19),
said set of valves being adapted to causing a peristaltic effect pumping of said fluid of interest circulating in said network of microfluidic channels from said set of microfluidic accesses to at least one sink (21) connected to said network of microfluidic channels.

2. Surface pump according to claim 1, characterised in that said network of microfluidic channels comprises a set of separate microfluidic drainage channels (5) provided with said set of microfluidic accesses, each microfluidic drainage channel being associated with a determined group of valves, and in that it comprises a single microfluidic actuating circuit (13) adapted to being connected to a single pressure generator (15), said actuating circuit being constituted of resistive and capacitive fluidic elements defining a response time delay between two successive valves belonging to a group of valves, said response time delay being configured to cause, under an action of said pressure generator, a sequential actuation of the valves.

3. Surface pump according to claim 2, characterised in that said set of microfluidic drainage channels comprises a set of main microfluidic channels (51) and a plurality of secondary microfluidic channels (53), each microfluidic drainage channel comprising a main microfluidic channel and a set of secondary microfluidic channels connecting the main channel to the associated microfluidic accesses (9).

4. Surface pump according to claim 2, characterised in that said microfluidic actuating circuit is formed of a coil-shaped microfluidic actuating channel, called actuating coil (113), having a characteristic length L between two successive valves belonging to a same group of valves, a width w, a height h, and a deformation parameter A, bearing out the following double inequality:
$$1 \times {10}^{- 3} \leq A \frac{{wL}^{2}}{{h}^{3}} \leq 10$$
and preferably
$$0.01 \leq A \frac{{wL}^{2}}{{h}^{3}} \leq 1.$$

5. Surface pump according to claim 2, characterised in that said microfluidic actuating circuit comprises comb-shaped actuating channels (33) defining capacitive elements, connected to a connection channel (35) defining a resistive element, the valves (19) being located on said actuating channels.

6. Surface pump according to claim 3, characterised in that the downstream ends (27a) of said main microfluidic channels are connected to a same sink, the other ends (27b) being closed.

7. Surface pump according to claim 3, characterised in that the set of main microfluidic channels is constituted of separate first and second sub-assemblies of main microfluidic channels (51a, 51b), the first sub-assembly of main microfluidic channels being connected to a first sink (21a) and the second sub-assembly of main microfluidic channels being connected to a second sink (21b).

8. Surface pump according to claim 2, characterised in that said pressure generator is configured to generate pressure slots to actuate in a cyclical manner the valves belonging to each group of valves.

9. Surface pump according to claim 1, characterised in that said network of microfluidic channels comprises a spiral-shaped main microfluidic channel (151) connected via secondary microfluidic channels (153) to said set of microfluidic accesses (9), and in that it comprises at least three separate microfluidic actuating circuits (313a, 313b, 313c) adapted to be connected to at least three separate pressure generators, each actuating circuit being constituted of a spiral-shaped actuating channel, said at least three actuating channels cooperating with each secondary microfluidic channel to form at least three valves (19a, 19b, 19c) configured to be actuated sequentially by said at least three pressure generators.

10. Surface pump according to claim 1, characterised in that it comprises a first substrate (41), a deformable membrane (43), and a second substrate (45), said at least one actuating circuit (13) being delimited by said deformable membrane and said first substrate, said network of microfluidic channels (5) being delimited by said deformable membrane and said second substrate, said network of microfluidic channels and said at least one actuating circuit then being separated by said deformable membrane at the levels of the intersecting zones thus forming a valve at each intersection.

11. Surface pump according to claim 1, characterised in that it comprises a determined number of valves comprised between around 10 and 1000.

12. Pumping system comprising a surface pump according to claim 1, further comprising at least one pressure generator connected to said at least one actuating circuit of said surface pump.

13. Dressing comprising a surface pump according to claim 1, characterised in that it comprises at least one sink connected to said surface pump, said at least one sink being intended to collect the fluid of interest pumped by the surface pump.

14. Dressing for exudative wounds, characterised in that it comprises a set of microfluidic drainage channels (5) provided with a plurality of microfluidic accesses (9) defining a predetermined domain adapted to covering a wound secreting exudate, a microfluidic actuating circuit (13) adapted to being connected to a single pressure generator (15), said microfluidic actuating circuit intersecting said microfluidic drainage channels on a plurality of deformable membrane intersecting zones forming a set of valves (19) adapted to causing, under an actuation of said pressure generator, a peristaltic effect pumping of the exudate circulating in said microfluidic drainage channels from said set of microfluidic accesses to at least one sink (21) connected to said set of microfluidic drainage channels.

15. Dressing according to claim 14, characterised in that said microfluidic actuating circuit is formed of a coil-shaped microfluidic actuating channel.

16. Dressing according to claim 14, characterised in that said microfluidic actuating circuit comprises comb-shaped actuating channels (33a, 33b) defining capacitive elements, connected to at least one connection channel (35) defining a resistive element, the valves being located on said actuating channels.

17. Dressing according to claim 16, characterised in that said set of microfluidic drainage channels comprises a set of main microfluidic channels (51) and a plurality of secondary microfluidic channels (53), each microfluidic drainage channel comprising a main microfluidic channel and a set of secondary microfluidic channels connecting the main channel to the associated microfluidic accesses.

18. Dressing according to claim 17, characterised in that it comprises first and second sinks (21a, 21b) for collecting the exudates secreted by the wound, and in that said set of main microfluidic channels is constituted of separate first and second sub-assemblies of main microfluidic channels (51a, 51b), the first sub-assembly of main microfluidic channels being connected to the first sink (21a) and the second sub-assembly of main microfluidic channels being connected to the second sink (21b).

19. Dressing according to claim 14, characterised in that the set of microfluidic drainage channels and the actuating fluidic channel are made of biocompatible material.

20. Dressing according to claim 14, characterised in that it comprises a support layer (81) receiving the set of microfluidic drainage channels and the actuating fluidic channel, said support layer being formed of a biocompatible porous material intended to be in contact with the wound.

21. Dressing according to claim 14, characterised in that it comprises an external self-adhesive layer (83).
